(19) United States
(12) Patent Application Publication
(10) Pub. No.: US 2014/0187857 A1
(43) Pub. Date: Jul. 3, 2014

(54) Apparatus and Methods for Enhanced Visualization and Control in Minimally Invasive Surgery

(71) Applicants: Jason Tomas Wilson, Santa Ana, CA (US); Vacit Arat, La Canada Flintridge, CA (US); Mark Scott Blumenkranz, Portola Valley, CA (US)

(72) Inventors: Jason Tomas Wilson, Santa Ana, CA (US); Vacit Arat, La Canada Flintridge, CA (US); Mark Scott Blumenkranz, Portola Valley, CA (US)

(73) Assignee: Vantage Surgical Systems Inc., Altadena, CA (US)

(21) Appl. No.: 13/761,136

(22) Filed: Feb. 6, 2013

Related U.S. Application Data
(60) Provisional application No. 61/595,467, filed on Feb. 6, 2012.
(60) Provisional application No. 61/622,922, filed on Apr. 11, 2012.

(51) Int. Cl. A61B 1/313; A61B 1/04; A61B 1/00; H04N 13/02; H04N 13/04

(52) U.S. Cl. CPC A61B 1/3132; H04N 13/0203; H04N 13/0452; H04N 13/0402; H04N 13/0429; A61B 1/00052; A61B 1/04; A61B 1/00009; A61B 1/00193; A61B 1/00188; A61B 1/00039
USPC 600/103

(57) ABSTRACT

Embodiments of the present invention provide improved visualization systems and methods for minimally invasive surgery. Some embodiments include the use of reverse kinematic positioning of camera systems to provide rapid and manual surgeon controllable positioning of camera systems as well as display of 3D surgical area images along the line of sight between a surgeon's eyes and the surgical area itself.

### RELATED APPLICATIONS

This application claims benefit of U.S. Provisional Application No. 61/595,467 filed Feb. 6, 2012, 61/622,922 filed Apr. 11, 2012, 61/693551 filed Aug. 27, 2012 and, 61/694678 filed Aug. 29, 2012 and is a Continuation-in-Part of U.S. patent application Ser. No. 13/268,071, filed Oct. 7, 2011. Each of these referenced applications is incorporated herein by reference as if set forth in full herein.

### FIELD OF THE INVENTION

The present invention relates generally to the field of minimally invasive surgery (MIS) and more particularly to enhanced visualization methods and tools for use in such surgical procedures.

### BACKGROUND OF THE INVENTION

Visualization of the surgical field during minimally invasive surgical procedures is indirect which can make the experience unintuitive and ergonomically incorrect on many levels. While during open surgery the surgeon looks directly at where his/her hands and instruments are; works in line with his/her visual axis with natural depth perception and peripheral vision. Duplicating this experience in MIS has never been achieved. To capture the images of the surgical field in real-time, surgeons have to rely on endoscopes which are inserted into the body close to the surgical field. These images are then displayed on monitors which are usually placed away from the sterile field and often in a direction not aligned with the motor axis of the surgeon during surgery. Furthermore, the surgeon's eyes are accommodated at a distance much farther than the work area which exacerbates a mental confusion which contributes in part to incorrect depth perception.

There have been many initiatives by visualization system manufacturers to improve this experience, the most impactful one of which has been the move to high-definition (HD) cameras and monitors. This, along with advancements in digital image processing, is reported widely to have made a big difference in the quality of visualization. The other major initiative has been to replace the 2-D cameras and monitors with 3-D versions addressing directly the problem of depth perception. While it was demonstrated that 3-D cameras and monitors improve surgeon performance in several critical tasks such as suturing, adoption has been limited due to surgeon reluctance for wearing special glasses (usually dark) in the operating room to watch such monitors, as well as various forms of discomfort (dizziness, fatigue, etc.) that results from doing so while standing up and performing many tasks for hours as necessary.

Another major difference between MIS and open surgery is that during MIS the surgeon needs someone else's help—often full-time—to see and navigate the surgical field. An assistant (surgeon assistant, attending nurse, etc.) holds and steers the endoscope under the surgeon's verbal instructions such that it can be directed to the desired area with the desired level of magnification and detail. Some robotically steered systems have also been introduced which are steered by voice or manual inputs; however these systems are expensive to use and maintain, and not commonly adopted.

To achieve high quality magnified views of the surgical field, zooming is performed by the assistant (or the robot) by physically moving the endoscope closer to the field. Some endoscope cameras may also incorporate limited optical zooming such as 2× integrated into the endoscope camera itself, but this is usually not enough to go from a full panoramic view of a body cavity to a highly detailed magnified view. Digital zoom is also an option; however, this approach suffers from subsampling and is not preferred. Regardless, however, only one view is available to the surgeon at any given time: a zoomed-out view where he/she can see the entire field including instrument coming in and out, or a magnified close-up view of the exact location of the surgery.

### SUMMARY OF THE INVENTION

Some embodiments of the invention are intended to address one or more of the above noted fundamental problems associated with visualization systems used in conventional minimally invasive surgery. In the prefferred embodiment these problems are addressed by providing the surgeon two or more views (e.g. panoramic top-level and magnified views) of the surgical field simultaneously in a picture-in-picture format.

In other embodiments, placement of the display is in the sterile field above the patient at an ergonomically correct eye-accommodation distance and orientation such that the surgeon's visual axis is in alignment with his/her motor axis.

In yet other embodiments, an auto-stereoscopic (glasses-free) screen is used which can operate in 2-D as well as 3-D modes based on user commands.

In some embodiments, the images on the screen can be manipulated by the surgeon through touchscreen commands which allow him/her to zoom in, zoom out, change picture-in-picture settings, and convert from 2-D to 3-D modes among other functions. This ability of the surgeon to control most if not all of the major facets of his/her visualization may eliminate the need for an assistant to steer the , thus saving costs and improving productivity.

In all embodiments, magnification of images is achieved by using optical means which allows the image resolution to remain the same high quality regardless of the zooming level.

In all embodiments, the images are captured via a single percutaneous lens inserted through an incision whose length is such that its tip stays as far from the surgical field as possible and at a stationary position: This minimizes the intrusion into the body space as well as the likelihood of contact with tissue, which is in direct contrast to a conventional endoscope the tip of which needs to be moved closer to the surgical are in order to capture zoomed-in images, which in the process may unintentionally cauterize such tissue.

In some embodiments, an ancillary benefit of the monitor repositioning is a larger field of view.

Improved visualization methods and apparatus of the various embodiments of the invention are applicable to many types of minimally invasive surgery, for example in the areas of laparoscopic, thoracoscopic, pelviscopic, arthroscopic surgeries. For laparoscopic surgery, significant utility will be found in cholecystectomy, hernia repair, bariatric procedures (bypass, banding, sleeve, or the like), bowel resection, hysterectomy, appendectomy, gastric/anti-reflux procedures, and nephrectomy.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single embodiment or aspect of the invention even though that may be the case with regard to some embodiments or aspects.

In a first aspect of the invention a percutaneous visualization system for providing a plurality of indirect views of a surgical area through a single incision, the system including a percutaneous lens assembly with a proximal end, a distal end, and one or more optical lenses in between, which is placed through an incision in a patient's body such that the proximal end is outside of the patient's body while the distal end disposed inside of the body cavity and aligned such that it is facing the surgical area a plurality of optical zoom lens assemblies each of which is aligned with the optical path of the percutaneous lens assembly, and each with a distal end and a proximal end and one or more movable lenses in between, wherein the distal end of each such zoom lens assembly receives the light emanating from the proximal end of the percutaneous lens, and directs the zoomed light through the proximal end of each such zoom lens assembly to an electronic capture means, wherein the magnification level of each of the zoom assemblies is independently controlled by user input, an electronic image capture means, comprising an at least one photosensitive integrated circuit, wherein the at least one photosensitive integrated circuit converts light exiting each zoom lens assembly to electrical image signals, an electronic processing means for formatting the electrical image signals from the at least one photosensitive integrated circuit for display on a display device based on user input, an at least one display device which receives the formatted electrical image signal and displays it for selectively viewing by the surgeon in two-dimensional or three-dimensional formats based on user input.

In a second aspect of the invention a surgical area viewing method for use in a minimally invasive surgical procedure, wherein the viewing method includes, making at least one percutaneous incision in the body of the patient in proximity to the surgical area, inserting at least one percutaneous lens assembly into the incision such that the proximal end of the lens assembly is outside of the patient's body while the distal end is disposed inside of a body cavity and aligned such that it is facing the surgical area, aligning at least one optical zoom assembly proximal to and in the optical path of the percutaneous lens, aligning at least one electronic image capture means in the optical path of the optical zoom assembly, wherein the electronic image capture means comprises one or more photosensitive integrated circuits, wherein the photosensitive integrated circuits convert light exiting each zoom lens assembly to electrical image signals, formatting the electronic image signals for display on a display device, viewing the formatted electronic image signals on a display device for viewing by the surgeon in two-dimensional or three-dimensional formats based on user input, manipulating a user input wherein the optical zoom assembly magnification level and electronic image formatting are chosen based on the user input.

In a third aspect of the invention a percutaneous visualization system for use in a minimally invasive surgical procedure for providing indirect views of a surgical area through a single incision, the system includes a percutaneous lens assembly with a proximal end, a distal end, and one or more optical lenses in between, which is placed through an incision in a patient's body such that the proximal end is outside the patient's body while the distal end disposed inside of a body cavity and aligned such that it is facing the surgical area, an optical zoom lens assembly which is aligned with the optical path of the percutaneous lens assembly, and with a distal end and a proximal end, and one or more movable lenses in between, wherein the distal end of such zoom lens assembly receives the light emanating from the proximal end of the percutaneous lens, and directs the zoomed light through the proximal end of such zoom lens assembly to an electronic capture means, wherein the magnification level is controlled by user inputs, an electronic image capture means, comprising an at least one photosensitive integrated circuit, wherein the at least one photosensitive integrated circuit converts light exiting each zoom lens assembly to electrical image signals, an electronic processing means for formatting the electrical image signals from the at least one photosensitive integrated circuit for display on a display device based on user input, an at least one display device which receives the formatted electrical image signal and displays it and facilitates touch screen inputs.

Other aspects of the invention will be understood by those of skill in the art upon review of the teachings herein. Other aspects of the invention may involve combinations of the above noted aspects of the invention. These other aspects of the invention may provide various combinations of the aspects presented above as well as provide other configurations, structures, functional relationships, and processes that have not been specifically set forth above.

### BRIEF DESCRIPTION OF THE DRAWINGS

FIG. 1 shows a cross sectional view of a first embodiment of a percutaneous lens placed in a percutaneous incision.

FIG. 2 shows a cross sectional view of a second embodiment of a percutaneous lens placed plug/trocar inserted in a percutaneous incision.

FIG. 3 shows a cross sectional view of a multi optical channel percutaneous image acquisition module.

FIG. 4 shows a schematic of two identical independent optical zoom assemblies.

FIG. 5 shows an example of a preferred embodiment of the display with a picture in picture view and touch screen inputs.

FIG. 6 is the surgeon's view of the display, multi optical channel percutaneous image acquisition module and patient.

FIG. 7 illustrates the advantage of placing the 10.1″ display in the sterile field, specifically providing a larger viewing area as compared to typical wall mounted surgical displays.

FIG. 8 is a block diagram illustrating the components of the invention.

FIG. 9 is a state machine diagram describing the control algorithm that manages the independent optical zoom assemblies and pixel wise image combinations.

### DETAILED DESCRIPTION OF THE PREFERED EMBODIMENS OF THE INVENTION

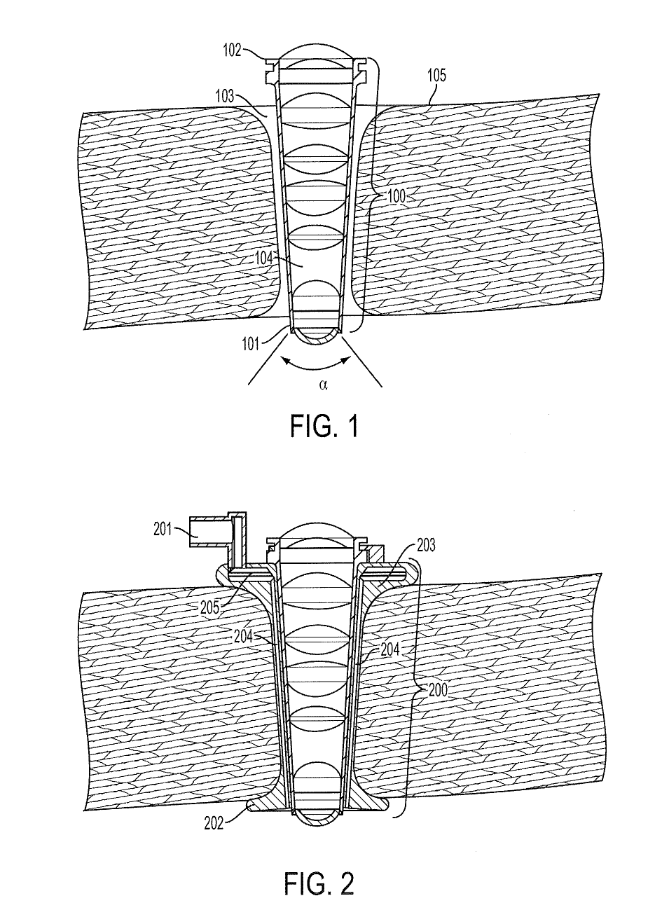

FIG. 1 provides a cross sectioned view of a first embodiment of a percutaneous lens 100 placed in an incision 103. The percutaneous lens has a distal end 101 which extends beyond the incision into a cavity above the surgical area (not shown). The percutaneous lens has a proximal end 102 that extends beyond the incision towards the surface of the patient's skin. The distal end and proximal end of the percutaneous lens are connected by a percutaneous optical channel 104. Disposed in the optical channel is an at least one lens element to gather light from the surgical field within some viewing angle a and direct it out beyond the incision proximal to the skin of the patient 105. Thus the proximal end of the percutaneous lens 102 must extend substantially to or beyond the patient's skin for the light to be directed out of the surgical cavity.

FIG. 2 shows another embodiment of the percutaneous lens 100 placed in a percutaneous incision 103. In this alternative embodiment the percutaneous lens 100 is inserted in a trocar or plug 200 that is placed in the incision 103. There are at least two methods to insert the lens 100 and plug 200 in the incision 103. The plug 200 may be inserted first following the insertion of the lens 100. Or the lens 100 and plug 200 may be assembled prior to insertion of the combination into the incision 103. To retain the plug in the incision, there may be flanges or elongations that extend in a direction perpendicular to the percutaneous lens 100. These elongations could be located distally 202, proximally 203, or both.

A one or more channels 204 may extend through the plug and/or the percutaneous lens 100, the distal end of which open to the body cavity and the proximal end of which connect to a common chamber or manifold 205. This manifold 205 is coupled to a connector means 201 to allow gas or fluid to be introduced into the body cavity. In a preferred embodiment, the body cavity is the gastrointestinal peritoneum and the gas is carbon dioxide for insufflation. However the fluid nor-anatomy are limited to either.

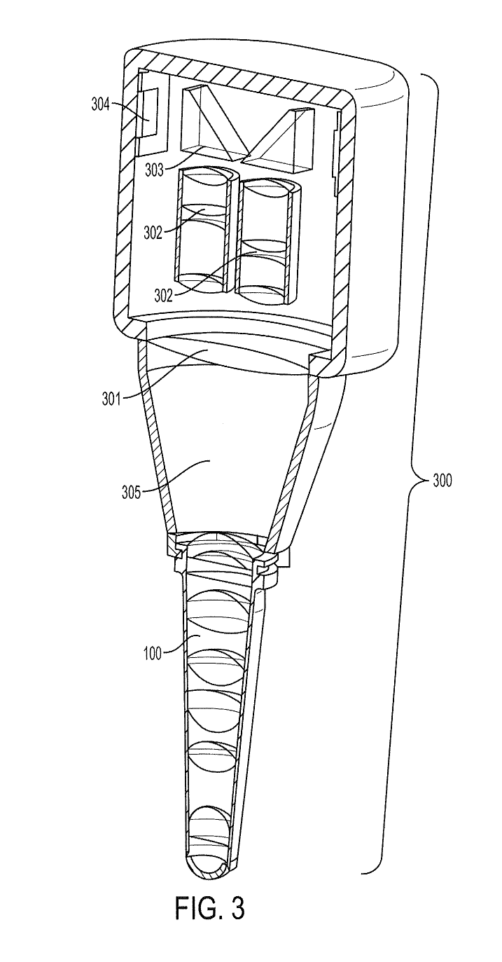

FIG. 3 shows a cross section of a multi optical channel percutaneous image acquisition module. The module consists of the percutaneous lens 100, intermediate optics 301, a plurality of independent optical zoom assemblies 302 in the optical path of the percutaneous lens, post zoom optics 304 and at least one photo sensor 305. As shown in the previous figures, the percutaneous lens is inserted into an incision in the patient. The intermediate optics 301 are aligned with the optical axis of the percutaneous lens. The intermediate optics 301 and post zoom optics 303 may consist of lenses, prisms, beam splitters and the like. Also in the preferred embodiment the body cavity is illuminated by Light Emitting Diodes (LED) lights, xenon lights or the like.

In the preferred embodiment, the plurality of optical zoom assemblies 302 include at least two laterally separated independent identical optical zoom assemblies with optical axes corresponding to left and right stereoscopic views. The optical zoom assemblies are independently zoomed allowing for each assembly to have different magnification powers simultaneously based on independently moving lens elements in each. Thus one may image a magnified view while the other provides a more wide angled view. Since the optics are identical and laterally separated, the zoom magnification can be synchronized to obtain simultaneous left and right identically magnified images corresponding to stereoscopic viewing by the left and right eye. The left and right images can be acquired by the at least one photo-sensor and displayed to the viewer for stereoscopic visualization. The amount of lateral separation between the independent optical zooming assemblies in part defines the amount of parallax that is perceived by a viewer.

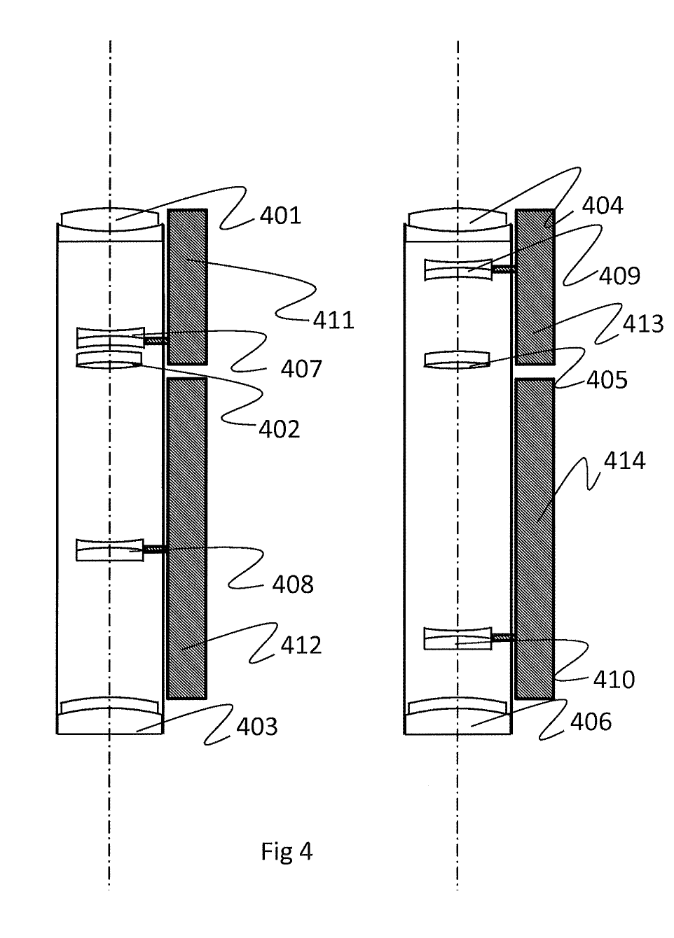

FIG. 4 shows an embodiment of the at least two independent identical optical zoom assemblies. Lens elements 401, 402, 403, 404, 405, and 406 ar stationary, while 407, 408, 409, 410 are movable in the direction of the optical axis. The optical axis is denoted by the dashed line. Although they are identical they are independent, as illustrated by the different locations of the movable lens elements. In the shown embodiment there are actuators 411, 412, 413, and 414 that move each movable lens element, the position of which dictate the magnification level of each independent zoom assembly. The movable lens elements can be guided by rails with an individual actuator for each, or they can be actuated simultaneously by a rotating barrel with a pin/slot guide which is commonly used in single lens reflex (SLR) cameras. Both zoom methods are common and well known in the art.

The actuators can be servo control motors with sensors, piezoelectric actuators with sensors, or stepper motors. Although two optical zoom lens assemblies are shown, there could be more than two. In the preferred embodiment there are at least two optical zoom assemblies that are laterally separated to provide either two 2D views of the surgical site with different magnifications, or they can be coordinated with the same magnification, each lens corresponding to the right and left eye of a stereoscopic view.

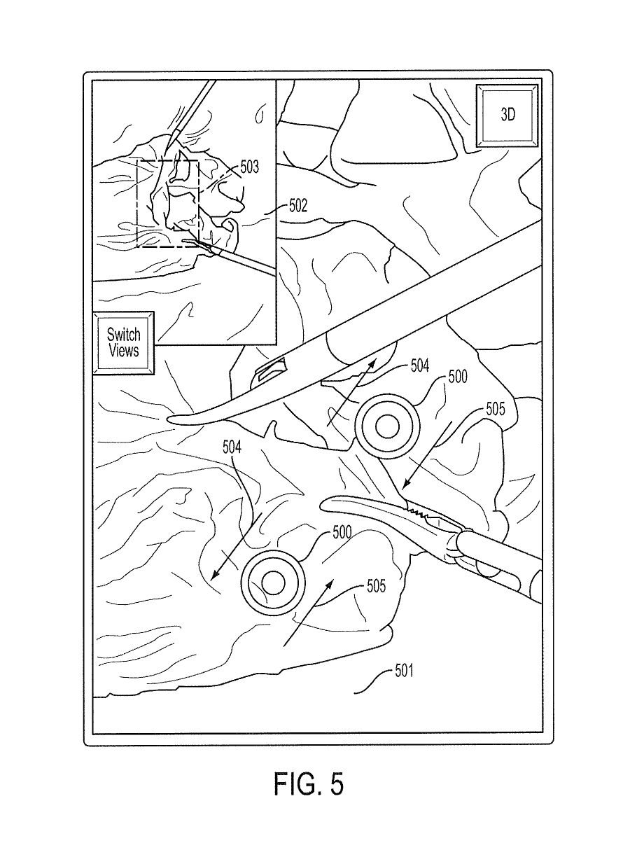

FIG. 5 shows an embodiment of the imaged surgical area by the multi optical channel percutaneous image acquisition module. In the shown embodiment there are two independent views. As shown the images are combined in a picture in picture manner. The full screen video image 501 is a magnified view of the surgical area, while the inlayed view 502 corresponds to a wider angled view. These views are available simultaneously as they are imaged by the independent zoom assemblies. The white square 503 denotes the region of the wider angle view that has been magnified and displayed as the main view 501. Although in this preferred embodiment there are two views, the invention is not limited to two views, and in general has more than two views. As shown, these views are combined in some pixel wise fashion to produce the enhanced visualization to the surgeon.

By pixel-wise it is meant that each pixel can be displayed from any image produced by the independent optical channels. As shown in FIG. 5 the pixel wise combination produces a picture in picture display. However in general the pixel wise combination could be column wise combinations, row wise combinations, or the like. The images could also be temporally interlaced, alternating each frame from a different optical channel. These types of combinations are particularly useful for 3D viewing. For active glasses or shutter type glasses, each image corresponding to the left and right eye shown in an alternating fashion. The active shutter type glasses allow the left and right eye to see the corresponding temporally interlaced left and right view in an alternating fashion. Typically this is done at a frame rate of 120 Hz as to make the switching imperceptible to the viewer. For passive glasses, the left and right images are also shown in sequence, each with a different light polarization that is filtered by the passive glasses. In the preferred embodiment the display is auto-stereoscopic employing a parallax barrier or a lenticular display.

In the preferred embodiment the display is touch screen. This allows the surgeon to manipulate the views via touch screen inputs. In the preferred embodiment the touch screen is capable of detecting multiple touch locations. As shown in FIG. 5, the bulls eye graphics 500 correspond to two distinct touch locations. If the user spreads his/her fingers relative to each other in the direction denoted by 504, the zoom lens assembly is commanded to optically magnify the view by physically moving lens components in the appropriate manner. If the user pinches his/her fingers together in the direction denoted by 505, the image is optically de-magnified by moving the lens components appropriately. The images can also be magnified/demagnified digitally as is common in digital photography.

Similarly the pixel-wise image combinations can also be changed by appropriate touch inputs on the display. For example the location of the inlay 502 could be moved by touching the inlay and dragging it to a new location. Furthermore the size can be adjusted as well by appropriate inputs. Menus and buttons can also be used to change viewing modalities. In the preferred embodiment the display is auto-stereoscopic and the 2D to 3D transition can also be controlled by touch inputs.

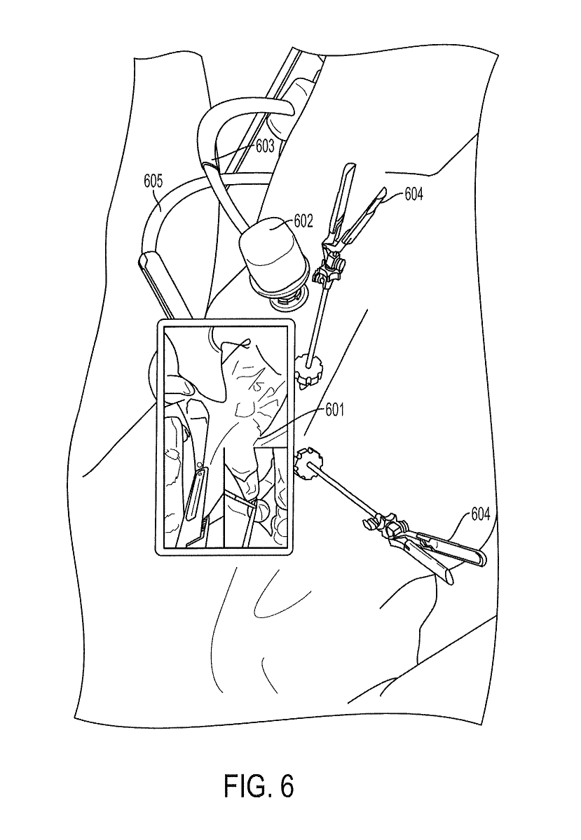

FIG. 6 is a view of the invention as seen by the surgeon. In the preferred embodiment the display 601 is positioned in the sterile field. The intent is to create a visualization experience that is as close to open surgery as possible. The multi optical channel percutaneous image acquisition module 602 is held by a steering arm 603. In one embodiment the arm may be robotic and steered using inputs via the touch screen or other input means. In alternative embodiments the arm may be locking, allowing the surgeon to move the arm when unlocked, while maintaining the stereo and rigid when locked. Thus maintaining the field of view when not adjusting multi optical channel percutaneous image acquisition module. The display is also held by and arm 605 and positioned so as to be close enough to the surgeon to operate the touch screen and/or hardware buttons, yet not obstructing the surgical instruments 604.

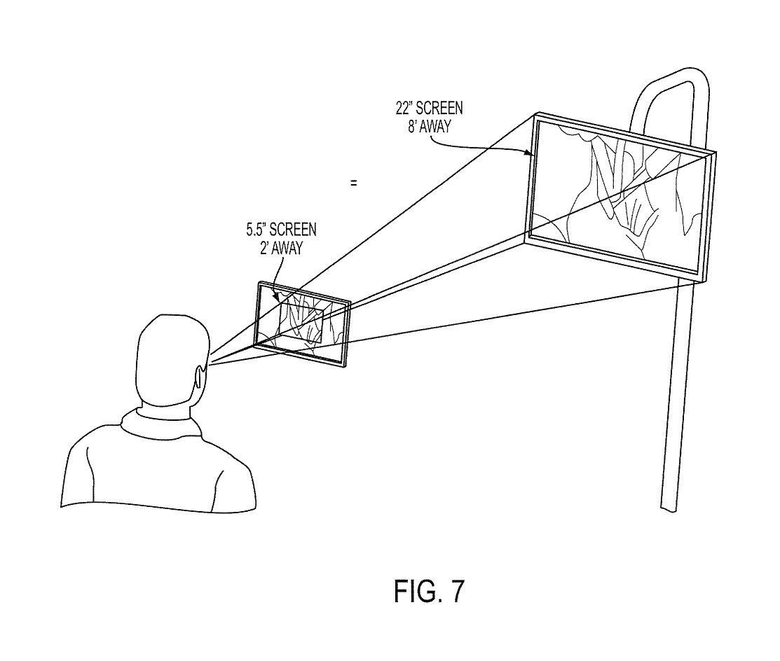

FIG. 7 illustrates an advantage gained by moving the screen into the sterile field. Typically laparoscopic surgery is done using monitors approximately 22 inches in the diagonal dimension placed approximately 8 feet away. Assuming a projective model, this is equivalent to a 5.5 inch display placed two feet away. Thus a 10.1 inch screen placed in the sterile field approximately two feet from the surgeon has the advantage of a larger viewing area, while allowing the surgeon to accommodate his/her eyes at a distance consistent with open surgery.

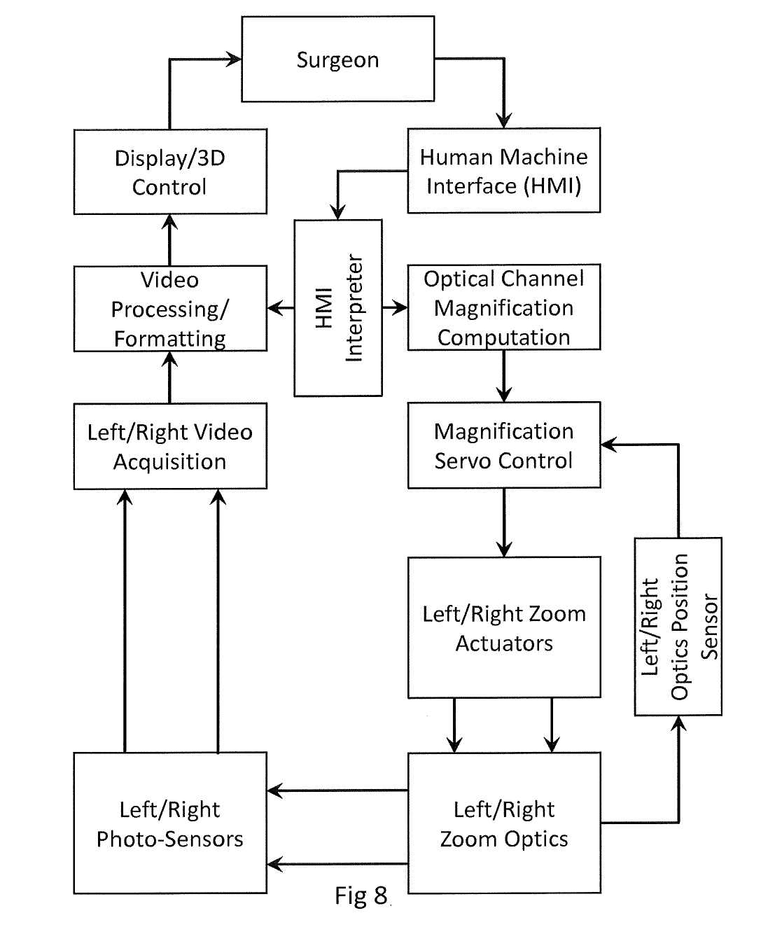

FIG. 8 is a block diagram showing the interconnection of various components of the invention. The surgeon controls the invention using the human machine interface (HMI). In the preferred embodiment the HMI is a touch screen interface. However the HMI can also consist of physical buttons, voice control, a camera with human feature recognition and the like.

An HMI interpreter algorithm analyzes the inputs from the surgeon. As an example, if the surgeon input is intended to activate the stereoscopic 3D view, the HMI interpreter analyzes the inputs as such. Based on these inputs the HMI interpreter sends commands to the display and/or the multi optical channel percutaneous image acquisition module. In the case of the display, these commands can trigger 2D to 3D transitions, pixel wise combining schemes (e.g. picture in picture), and display settings (e.g. brightness), and the like. In the case of the camera, this can be in the form of zoom/magnification commands, standard camera settings (e.g. gain, exposure, white balance), etc. Once the HMI interpreter interprets the surgeon inputs, the data is sent to the optical channel magnification computation. In a preferred embodiment, if a pinch or spread gesture is read as shown in FIG. 5, the change in magnification is computed as proportional to the relative motion of the two touch locations.

Once the magnification level is computed, the reference signal is sent to the magnification servo control. The magnification servo control is a computer algorithm that regulates the position of the optical components of the independent optical zoom assembly. In a feedback mode the servo control algorithm reads the optics position sensor and regulates the optics position by sending commands to the zoom actuators. In a preferred embodiment there are an at least two independent optical zooming mechanisms corresponding to the left and right eye of a stereoscopic camera. When in stereoscopic mode, the left and right zoom actuators are sent commands based on the left/right optics position sensors to keep the magnifying power the same, thus imaging the surgical area in a stereoscopic fashion. In a 2D or picture in picture modality, the two optical channels image a magnified and wider angle view of the surgical site.

In the preferred embodiment, the light from the at least left/right zoom optics are focused onto the left/right photosensors. The left/right photosensors could be one sensor for the at least left/right optical channels or a plurality of sensors. If a plurality of sensors is used, additional optical components may be used to direct specific bands of light wavelengths to each sensor. After the light is measured by the photosensors, image acquisition electronics convert the photosensor charges to digital image information and are sent to the video processing/formatting electronics. These electronics perform all or some of image sharpening, color correction, pixel wise formatting (e.g. picture in picture), up-sampling, down-sampling and the like, as well as pixel wise formatting including but not limited to spatial and temporal interleaving of pixel data. This can manifest as simply interleaving frames from each optical channel, or combining frames in a pixel wise fashion to create picture in picture views, alternating image columns and the like. The processed/formatted images are sent to the display, as well as other configuration data pertaining to settings such as parallax barrier on and off commands, infrared cuing for active glasses, and light polarization for passive glasses. Finally the video is displayed to the surgeon.

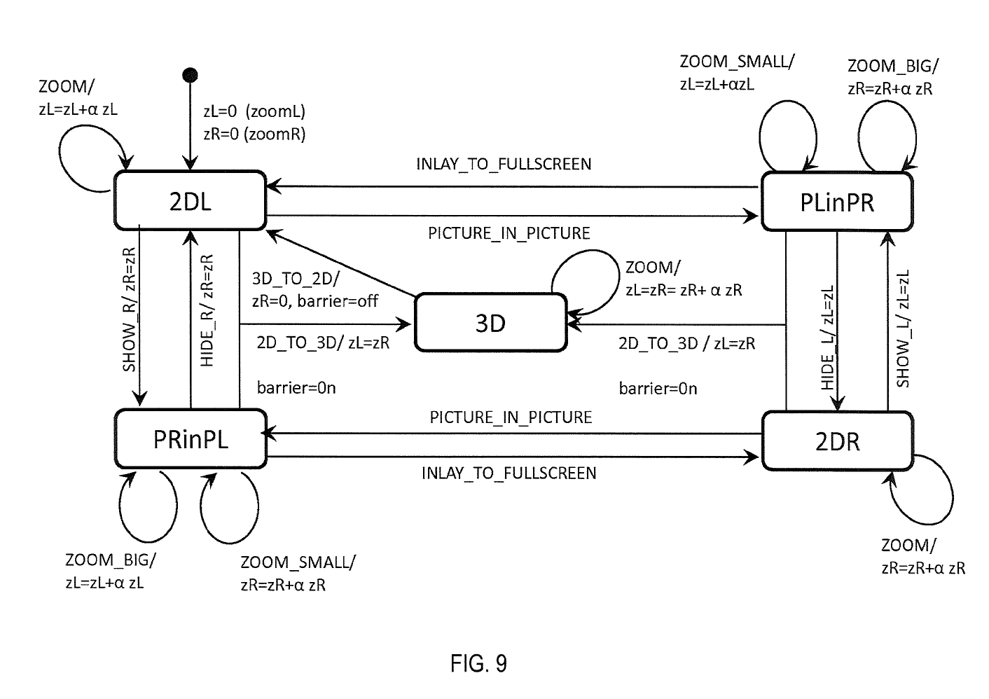

FIG. 9 shows a preferred embodiment of a state machine diagram that controls the at least two identical independent zoom assemblies that are laterally disposed from each other to facilitate either independent 2D viewing of the surgical area with different magnification, or coordinated identical magnification for stereoscopic image acquisition. The algorithm enters the state machine in a single 2D viewing mode using the left optical channel, denoted as 2DL. This is, without loss of generality as the right view could be chosen as well.

From the standard 2D view, the viewing modality can change to 2D picture in picture or to a 3D stereoscopic view based on the surgeon inputs. As shown, the picture in picture view can either be the left view inlayed on the right view or the right view inlayed on the left view. In the state machine diagram this is denoted by PRinPL for right channel inlayed on left channel, or PLinPR for left optical channel inlayed on right optical channel. From either picture in picture view, the view can be restored to 2DL, the view imaged by the left optical channel, or changed to 2DR denoting the view imaged by the right optical channel.

In the case of transitioning to 3D viewing, the two optical channel's magnification levels are synchronized and the left/right views are formatted appropriately to display the 3D view. In the case of active glasses, the left and right frames are shown in an alternating fashion synchronized with the shutters of the glasses. In the case of passive glasses, the left and right view are displayed with the appropriate light polarization, allowing the left and right eye to view the corresponding left and right view. In the preferred embodiment, the display is auto-stereoscopic showing the left and right view to each eye based on parallax barrier technology or a lenticular display.

During each state in the state machine diagram, the magnification level of each view can be adjusted based on user inputs. In the preferred embodiment the magnification level of each independent optical zoom assembly can be adjusted independently. During 2D viewing, either picture in picture or a single view, the magnification can be controlled for each optical zoom assembly independently. In the case of stereoscopic viewing or 3D, the magnification is synchronized.

The following paragraphs provide additional information about selected components and their functionality.

The functional purpose of the plug/trocar 202 is to hold the device down to the patient by an expanded flange. The plug must be deformable enough to allow insertion into the incision, either by the natural compliance of the material that it is constructed from, by being or having inflatable components, or having articulating components. In the preferred embodiment the plug is disposable, but at the minimum it is sterilizable.

The percutaneous lens assembly 100 may have optical components made of glass or plastic, however in the preferred embodiment it is disposable, but at the minimum it is sterilizable.

The coupler 305 must be rigid enough to maintain sufficient optical alignment between the percutaneous lens assembly 100 and the multi optical channel percutaneous image acquisition module 101. It must have means to attach to the percutaneous lens assembly 100 or plug 200 and means to attach to the steering frame 603 or multi optical channel percutaneous image acquisition module 300.

The multi optical channel percutaneous image acquisition module 300 contains numerous optical and electronic components of the system which may limit the ability for this unit to be treated as disposable in some embodiments and in such embodiments may instead be designed for multiple uses and the unit may be configured for ease of surface sterilizability. This unit typically includes optical zoom and focusing mechanisms, photosensitive integrated circuits, and digital image processing electronics. In some basic embodiments, two photosensitive integrated circuits, one associated with each pupil, and thus with each optical channel created by the stereoscopic pupils, may be the extent of the electronic components in the unit. However, to obtain better image quality and truer color, 3 or 4 photosensitive integrated circuits may be used to sense different wavelengths of light separately (e.g. red, green, and blue). In this case extra optical hardware may need to be added, such as dichroic prisms, in order to optically separate the different wavelengths of light. In still other embodiment variations, it may be desirable to sacrifice image quality for compactness, and use a single photo sensor to capture both right and left images, half for the left and half for the right. Zooming could be continuous, or could have a finite number of discrete zoom levels. Focus could be manual or automatic.

The display 601 communicates to the multi optical channel percutaneous image acquisition module 300 by wired, wireless or the like communication. This could be a single direction communication where the image data is simply sent to the display 601 for viewing. The display may also have touch screen controls for zoom, focus, image freezing, or other camera mode selections, requiring the communication between the devices to support two-way information flow. A touch screen interface could be button based or gesture based. For example, a gesture to zoom out would be to perform a two finger pinching motion on the screen and the picture-in-picture roles could be reversed by swiping from the smaller image to the center of the screen. The display 601 may support VGA resolution (640×480) all the way up to true high definition (1920×1080p) or beyond. Since the multi optical channel percutaneous image acquisition module 300 facilitates stereoscopic image acquisition, the display 601 preferably supports either active or passive 3D display technology. In the preferred embodiment, the display is auto-stereoscopic (e.g. parallax barrier), requiring no glasses for viewing a 3-D effect.

In some embodiments, the movement of the objective lens assembly may be largely rotational in nature such that the objective lens assembly pivots about the most distal lens or about the entry point of the assembly into the skin or other tissue of the patient. In other embodiments, movement of the assembly may be such that it undergoes some translation relative to the base and as such some repositioning of the base relative to the patient's skin may be used to ensure that undue stressing of the patient's tissue does not occur.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be solely limited by the claims presented hereafter.

1. A percutaneous visualization system for providing a plurality of indirect views of a surgical area through a single incision, the system comprising:
a percutaneous lens assembly with a proximal end, a distal end, and one or more optical lenses in between forming an optical path that can be aligned to the surgical area, wherein the proximal end is configured to be outside of a patient's body while the distal end is disposed inside of the patient's body;
the percutaneous lens assembly including a plurality of optical zoom lens assemblies each including a distal end and a proximal end and one or more movable lenses in between, wherein:
each of the plurality of optical zoom lens assemblies is configured to be aligned with the optical path of the percutaneous lens assembly,
at least one of the distal ends of the plurality of optical zoom lens assemblies is configured to receive light emanating from the proximal end of the percutaneous lens and direct the light through the proximal end of the at least one of the plurality of optical zoom lens assemblies to an electronic capture means, and
the magnification level of each of the zoom assemblies is configured to be independently controlled;
an electronic image capture device comprising an at least one photosensitive integrated circuit configured to convert light from at least one of the optical zoom lens assemblies to electrical image signals;
a processor configured to format the electrical image signals from the at least one of the photosensitive integrated circuits for display on a display;
one or more-display devices in communication with the electronic processing means and configured to receive and display the formatted electrical image signal(s).

2. The system of claim 1, wherein at least two of said plurality of the optical zoom lens assemblies have different magnification levels.

3. The system of claim 1, wherein at least two of said plurality of the optical zoom lens assemblies have magnification levels that are the same.

4. The system of claim 3 wherein at least two electronic image signals acquired from the at least two optical zoom assemblies are stereoscopic image pairs.

5. The system of claim 1 wherein at least one display device is a stereoscopic display device which requires stereoscopic viewing glasses for viewing.

6. The system of claim 1 wherein the at least one display device is an autostereoscopic display device which allows viewing without the use of any stereoscopic viewing glasses.

7. The system of claim 1 wherein the percutaneous visualization system is configured to be controlled through user inputs entered through a touch screen interface on the display device.

8. The system of claim 1 wherein the formatting comprises pixel wise combinations of at least two electronic images.

9. The system of claim 1 wherein the display device is configured to be placed in the sterile field.

10. The system of claim 9 wherein the viewing angle of the display device is configured to be aligned with the motor axis of the surgeon.

11. The system of claim 9 wherein the display device is configured to be placed at a distance that closely matches the distance of the actual surgical area relative to the surgeon.

12. The system of claim 7 wherein the touch screen inputs are configured to control the magnification levels of each of the optical zoom assemblies.

13. The system of claim 7 wherein the touch screen inputs are configured to control the pixel wise combination of images.

14. A method of viewing a surgical area during a minimally invasive surgical procedure, method comprising:
providing a percutaneous lens assembly configured to be inserted into an incision such that a proximal end of the percutaneous lens assembly can be outside of the patient's body while the distal end can be disposed inside of a body cavity and aligned such that its optical path can face the surgical area;
providing at least one electronic image capture device in the optical path of the percutaneous lens assembly including one or more photosensitive integrated circuits configured to convert light exiting the proximal end of the percutaneous lens assembly to electrical image signals;
communicating the at least one electronic image capture device with a processor configured to format the electronic image signals for display on a display device;
projecting the formatted electronic image signals on the display device for viewing in two-dimensional or three-dimensional formats based on a user's input;
manipulating one or both of the optical magnification level and the electronic image formatting according to the user's input.

15. The method of claim 14 wherein the step of providing the percutaneous lens assembly further comprises:
providing at least two optical zoom lens assemblies that can be configured to have magnification levels that are different from each other and forming part of the percutaneous lens assembly.

16. The method of claim 14 wherein the step of providing the percutaneous lens assembly further comprises:
providing at least two optical zoom lens assemblies that can be configured to have magnification levels that are the same and forming part of the percutaneous lens assembly.

17. The method of claim 16 wherein the step of providing at least one electronic image capture device further comprises:
acquiring from the at least two optical zoom assemblies stereoscopic image pairs.

18. The method of claim 14 wherein the step of projecting the formatted electronic image signals on the display device further comprises projecting the formatted electronic image signals on a stereoscopic display device which requires stereoscopic viewing glasses for viewing.

19. The method of claim 14 wherein the step of projecting the formatted electronic image signals on the display device further comprises
projecting the formatted electronic image signals on an autostereoscopic display device which allows viewing without the use of any stereoscopic viewing glasses.

20. The method of claim 15 additionally comprising: receiving the user's input through a touch screen interface on the display device.

21. The method of claim 14 wherein the communicating the at least one electronic image capture device with the processor further comprises:
formatting pixel wise combinations of at least two electronic images.

22. The method of claim 14 wherein the step of projecting the formatted electronic image signals further comprises:
locating the display device in the sterile field.

23. The method of claim 22 wherein the step of projecting the formatted electronic image signals further comprises:
providing a viewing angle of the display device that is aligned with the motor axis of a surgeon.

24. The method of claim 22 wherein the step of projecting the formatted electronic image signals further comprises:
configuring the display devicen to be placed of between the actual surgical area and the surgeon.

25. The method of claim 20 wherein the step of providing the at least two optical zoom level assemblies further comprises:
receiving touch screen inputs through the display device to control the magnification levels of each of the optical zoom assemblies.

26. The method of claim 20 wherein the step of proving the at least two optical zoom level assemblies further comprises:
receiving touch screen inputs through the display device to control the pixel wise combination of images.

27. A percutaneous visualization system for use in a minimally invasive surgical procedure for providing indirect views of a surgical area through a single incision, the system comprising:
a percutaneous lens assembly with a proximal end, a distal end, and one or more optical lenses in between forming an optical path configured to be placed through an incision in a patient's body such that the proximal end is outside the patient's body while the distal end is disposed inside of a body cavity;
an optical zoom lens assembly with its optical axis aligned with the optical path of the percutaneous lens assembly, the optical zoom lens assembly including a distal end and a proximal end, and one or more movable lenses in between configured to move relative to each other to change the magnification level based on an user's input;
an electronic image capture device comprising at least one photosensitive integrated circuit, wherein the at least one photosensitive integrated circuit is configured to convert light exiting the optical zoom lens assembly to electrical image signals;
a processor configured to format the electrical image signals from the at least one photosensitive integrated circuit for display on a display device based on the user's input;
an at least one display device which receives the formatted electrical image signal and displays it and facilitates touch screen inputs.

28. The system of claim 27 wherein the user inputs are entered through a touch screen interface on the display device.

29. The system of claim 27 wherein the display device is placed in the sterile field.

30. The system of claim 29 wherein the viewing angle of the display device is aligned with the motor axis of the surgeon.

31. The system of claim 29 wherein the display device is placed at a distance that closely matches the distance of the actual surgical area relative to the surgeon.

32. The system of claim 28 wherein the touch screen inputs control the magnification level of the optical zoom assembly.
